(19) United States
(12) Patent Application Publication
(10) Pub. No.: US 2021/0030654 A1
(43) Pub. Date: Feb. 4, 2021

(54) ANTIOXIDANT COMPLEX AND COMPOSITION FOR FORMING SAME

(71) Applicant: Soo Ahn HWANG, Seoul (KR)

(72) Inventors: Soo Ahn HWANG, Seoul (KR); Sung Hyun KIM, Seoul (KR); Vladimir STAROVEROV, Seoul (KR)

(73) Assignee: Soo Ahn HWANG, Seoul (KR)

(21) Appl. No.: 17/074,152

(22) Filed: Oct. 19, 2020

Related U.S. Application Data
(63) Continuation of application No. PCT/KR2018/009855, filed on Aug. 27, 2018.

(51) Int. Cl. A61K 8/49; A61K 8/34; A61Q 19/08

(52) U.S. Cl. CPC A61K 8/498; A61K 2800/49; A61Q 19/08; A61K 8/345

(57) ABSTRACT

The present invention is applied to the skin care cosmetic field and relates to an antioxidant complex that exhibits excellent antioxidant capacity within cells. An antioxidant complex according to an embodiment of the present invention may comprise flavonoid having an antioxidant efficacy, and a solubilizer solubilizing the flavonoid.

### CROSS-REFERENCE TO RELATED APPLICATIONS

The present application is a continuation of International Patent Application No. PCT/KR2018/009855, filed on Aug. 27, 2018. The disclosure of the above-listed application is hereby incorporated by reference herein in their entirety.

### BACKGROUND

Embodiments of the inventive concept described herein relate to a field related to antioxidant skin care cosmetics, and relates to an antioxidant complex that exhibits excellent antioxidant capacity in cells, and relates to an antioxidant complex containing flavonoid and a solubilizer, and to a composition constituting the complex.

Dihydroquercetin is flavonoid that enhances a protective function of a skin from external and internal toxicity, radiation, bacteria and other environmental factors, and that is applied topically, and, in particular, normalizes general metabolic processes of a lymphatic system and a blood system to alleviate skin aging.

Dihydroquercetin is a powerful antioxidant derived from a plant that may protect cells from harmful effects of excessive free radicals. Because of antibacterial and anti-inflammatory properties thereof, dihydroquercetin is used in cosmetics that cause skin problems.

These cosmetics containing the dihydroquercetin provide an alleviating effect to inflammatory and/or irritated skin to be changed to healthy skin (FEBS Letters, 520, 2002).

Dihydroquercetin inhibits melanin production and exhibits possibility of being used as a whitening substance (Phytotherapy Res., 22 (2008); KR10-0825628; KR10-0710657).

Dihydroquercetin which acts at a cellular level strengthens a capillary wall, stimulates cellular metabolism, and protects the skin from ultraviolet rays. Therefore, topical cosmetics containing the dihydroquercetin soften the skin and protect the skin from negative environmental influences.

Dihydroquercetin accelerates a wound healing process by promoting skin regeneration. Among natural antioxidants, a dihydroquercetin molecule has the highest activity, but does not exhibit a sufficient effect in vivo due to low solubility thereof in water or oil. Therefore, use of the dihydroquercetin in topical cosmetic formulations is limited due to the low solubility of flavonoid and low stability of a composition based on the same.

One of various approaches is chemical or enzymatic derivatization of a hydroxyl group with more lipophilic substituent. Chemical modification is non-selective and thus leads to a mixture of products with varying numbers of derivatives. In addition, a reaction must often be carried out under harsh reaction conditions such as toxic media or high temperatures (U.S. Pat. No. 9,822,104B2, JP4790561, JP4790561).

The complex containing dihydroquercetin and α- and β-cyclodextrin is characterized by high water solubility thereof, long-term action thereof in organisms, and stability during transport thereof in a bloodstream. Although use of the dihydroquercetin in a complex thereof with β-cyclodextrin which may act for several hours after infiltrating thereof into cells is more promising from a medical point of view than use of pure dihydroquercetin (J. Pharmacy and Pharmacology 3, 2015), the former has a therapeutical purpose.

Microemulsion was used as a delivery system for skin penetration as a method of improving the solubility of dihydroquercetin (10th World Meeting on Pharmaceutics, Biopharmaceutics and Pharmaceutical Technology).

Another approach to the use of dihydroquercetin is synthesis of polymers or hydrophobic fine particulates or nanoparticles (U.S. Pat. Nos. 7,067,152, 6,979,440).

Therefore, an approach for promoting the skin penetration by carrying an active ingredient on nanoparticles has been recently developed. This is known as a form of nanoemulsion in food, cosmetics and functional food groups. However, this nanocomplex contains a large amount of surfactant and is harmful to the skin. This system is unproductive because molecules are trapped in a particle matrix and thus release thereof is prolonged, thereby to delay absorption thereof by the skin due to an insufficient concentration of active flavonoid in the system.

### SUMMARY

Embodiments of the inventive concept provide a method of preparing a biologically available cosmetic additive containing dihydroquercetin in a form in which inherent antioxidant properties thereof is able to be exhibited in vivo. Further, embodiments of the inventive concept provide an antioxidant complex which may be prepared in a simple process is simple and contains a high concentration of dihydroquercetin, and provide a composition that constitutes the complex.

Other purposes and advantages of the inventive concept become more apparent from the detailed description, claims, and drawings of the disclosure.

According to an exemplary embodiment, a composition constituting an antioxidant complex contains flavonoid having antioxidant efficacy, and a solubilizer to solubilize the flavonoid.

The flavonoid includes dihydroquercetin, wherein the flavonoid is a mixture of the dihydroquercetin and one selected from a group consisting of armadendrin, eriodictiol, quercetin, naringenin, and pinocembrin depending on purification of an extract of the dihydroquercetin.

The solubilizer is one selected from a group consisting of butylene glycol, propylene glycol and glycerol.

A content of the flavonoid is 0.001 to 50% by weight, and a content of the solubilizer is 50 to 99.999% by weight.

The content of the flavonoid is 0.1 to 20% by weight, and the content of the solubilizer is 70 to 99.9% by weight.

The composition constituting the antioxidant complex further contains at least one selected from a group consisting of surfactant, pigment, stabilizer, emollient and humectant.

According to an exemplary embodiment, an antioxidant complex contains flavonoid having antioxidant efficacy, and a solubilizer to solubilize the flavonoid.

The flavonoid and the solubilizer are agglomerated with each other.

The flavonoid and the solubilizer are agglomerated with each other such that the solubilizer surrounds an outer periphery of the flavonoid.

The flavonoid includes dihydroquercetin, wherein the flavonoid is a mixture of the dihydroquercetin and one selected from a group consisting of armadendrin, eriodictiol, quercetin, naringenin, and pinocembrin depending on purification of an extract of the dihydroquercetin.

The solubilizer is one selected from a group consisting of butylene glycol, propylene glycol and glycerol.

A content of the flavonoid is 0.001 to 50% by weight, and a content of the solubilizer is 50 to 99.999% by weight.

The content of the flavonoid is 0.1 to 20% by weight, and the content of the solubilizer is 70 to 99.9% by weight.

The composition constituting the antioxidant complex further contains at least one selected from a group consisting of surfactant, pigment, stabilizer, emollient and humectant.

According to an exemplary embodiment, a cosmetic formulation contains an antioxidant complex, wherein the antioxidant complex contains flavonoid having antioxidant efficacy, and a solubilizer to solubilize the flavonoid.

Other specific details of the inventive concept are included in the detailed description and drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a graph showing a protective effect of an antioxidant complex;

FIG. 2 is a graph showing an experiment of an optimal protective effect of the antioxidant complex;

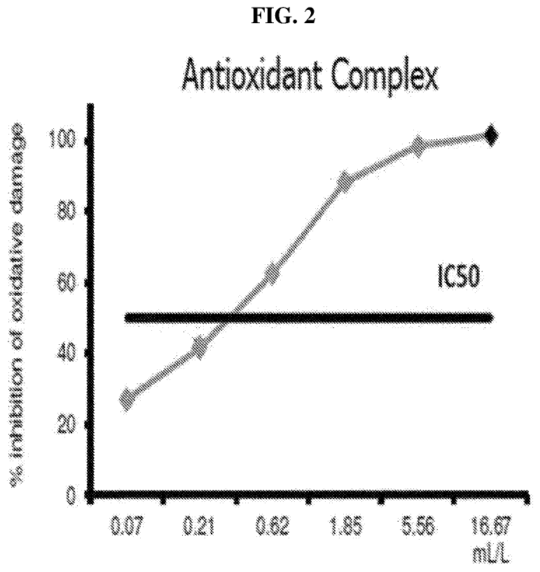

FIG. 3 is a graph analyzing Cap-e of the antioxidant complex;

FIG. 4 is a graph analyzing Cap-e of dihydroquercetin not constituting a complex; and

FIG. 5 is a graph analyzing Cap-e of each of bio-dihydroquercetin and a dihydroquercetin complex according to the inventive concept.

### DETAILED DESCRIPTION

Advantages and features of the inventive concept, and methods of achieving them will become apparent with reference to embodiments described below in detail in conjunction with the accompanying drawings. However, the inventive concept is not limited to the embodiments disclosed below, but may be implemented in various forms. The present embodiments are provided to merely complete the disclosure of the inventive concept, and to inform merely fully those skilled in the art of the inventive concept of the scope of the inventive concept. The inventive concept is only defined by the scope of the claims. exemplary embodiments of the inventive concept will be described in detail with reference to the accompanying drawings. Like reference numerals refer to like elements throughout the disclosure. As used herein, the term “and/or” includes any and all combinations of one or more of the associated listed items.

The terminology used herein is for the purpose of describing the embodiments only and is not intended to limit the inventive concept. As used herein, the singular forms “a” and “an” are intended to include the plural forms as well, unless the context clearly indicates otherwise. It will be further understood that the terms “comprises”, “comprising”, “includes”, and “including” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, a composition constituting an antioxidant complex, an antioxidant complex, and a cosmetic formulation containing the antioxidant complex according to embodiments of the inventive concept will be described.

First, the composition constituting the antioxidant complex according to an embodiment of the inventive concept may contain flavonoid having antioxidant efficacy and a solubilizer to solubilize the flavonoid.

The flavonoid may be extracted from, for example, Lalix sibirica wood as a coniferous tree that occupies a vast Siberian region. Lalix sibirica wood contains a variety of flavonoids. Such flavonoids may include dihydroquercetin, armadendrin, eriodictiol, quercetin, naringenin, and pinocembrin, etc. Among them, a content of dihydroquercetin is 90% or greater.

Therefore, the flavonoid of the composition constituting the antioxidant complex according to one embodiment of the inventive concept has a content of dihydroquercetin at a 90% or greater amount.

In addition, the flavonoid of the inventive concept is a mixture further containing at least one selected from a group consisting of armadendrin, eriodictiol, quercetin, naringenin, and pinocembrin according to purification of an extract of dihydroquercetin.

The solubilizer functions to solubilize the flavonoid in a solvent. The solubilizer may be one selected from a group consisting of butylene glycol, propylene glycol, and glycerol.

In one example, a content of the flavonoid may be 0.001 to 50% by weight, and a content of the solubilizer may be 50 to 99.999% by weight. Preferably, the content of the flavonoid may be 0.1 to 20% by weight, and the content of the solubilizer may be 70 to 99.9% by weight.

When the content of the flavonoid is lower than 0.001% by weight, the antioxidant effect of the flavonoid may not occur. To the contrary, when the content of flavonoid exceeds 50% by weight, the antioxidant effect may not be considerable compared to the excess content.

When the content of the solubilizer is lower than 50% by weight, it is difficult to secure emulsion stability of an oil phase component and an aqueous phase component, such that phase separation easily occurs. To the contrary, when the content of the solubilizer exceeds 9.999% by weight, a content of functional substances may be relatively smaller, thus making it difficult to perform a desired function, and causing skin irritation and other skin problems, which may be undesirable.

A content of dihydroquercetin contained in the composition constituting the antioxidant complex may be 0.001 to 50% by weight, and preferably, the content of dihydroquercetin may be 0.1 to 20% by weight.

When the solubilizer is, for example, butylene glycol, a content of butylene glycol contained in the composition constituting the antioxidant complex may be 50 to 99.999% by weight. Preferably, the content of butylene glycol may be 70 to 99.9% by weight.

In one example, when the solubilizer is, for example, propylene glycol, a content of propylene glycol contained in the composition constituting the antioxidant complex may be 50 to 99.999% by weight. Preferably, the content of propylene glycol may be 70 to 99.9% by weight.

In one example, when the solubilizer is, for example, glycerol, a content of glycerol contained in the composition constituting the antioxidant complex may be 50 to 99.999% by weight. Preferably, the content of glycerol may be 70 to 99.9% by weight.

The composition constituting the antioxidant complex may further contain at least one selected from a group consisting of surfactants, pigments, stabilizers, emollients, and humectants.

In this connection, the surfactant may be one selected from a group consisting of cetyl alcohol, stearyl alcohol, behenyl alcohol, lecithin, hydrogenated lecithin, isostearic acid, isostearyl alcohol and isotridecyl alcohol.

The pigment may be, for example, one of mica, talc, sericite, kaolin, and nylon powder.

The stabilizer may be one selected from a group consisting of epigallocarechin gallate (EGCG), butylated hydroxytoluene (BHT), butylated hydroxyanisole (BHA), gamma-glabridin (γ-glabridin), ferulic acid, propyl gallate, sodium metabisulfite, ubiquinone), tocopherol, lipoic acid, beta-carotene (β-carotene), resveratrol, and phytic acid.

The emollient may be one selected from a group consisting of dipentaerythritylhexahydroxystearate/hexastearate/hexaloginate, dipentaerythrityl tri-polyhydroxystearate, polyglyceryl-2isostearate/dimerdilinoleate copolymer, hydrogenated castor oil dimer dilinoleate, polyglyceryl-10 nonisostearate, phytosteryl/isostearyl/cetyl/stearyl/behenyldimerdilinoleate.

The humectant may be one selected from a group consisting of mono glycerin, diglycerin and triglycerin, propylene glycol, ethylene glycol, butylene glycol, dipropylene glycol, pentylene glycol, hexylene glycol and soribitol.

Subsequently, an antioxidant complex according to another embodiment of the inventive concept will be described. The antioxidant complex according to this embodiment is prepared as the composition constituting the antioxidant complex as above-described. Therefore, the antioxidant complex according to the present embodiment may be substantially the same substance as the composition.

The antioxidant complex according to the present embodiment is formed by thermally blending the composition constituting the antioxidant complex as above-described. More specifically, the flavonoid and the solubilizer are mixed with each other and the mixture is blended at 60 to 90° C. such that the flavonoid and the solubilizer are agglomerated with each other to prepare the antioxidant complex.

Accordingly, the antioxidant complex according to an embodiment of the inventive concept may have the flavonoid and the solubilizer in a form in which both are agglomerated with each other. More preferably, the flavonoid and the solubilizer may be agglomerated with each other such that the solubilizer surrounds an outer periphery of the flavonoid.

Next, a cosmetic formulation according to another embodiment of the inventive concept will be described. The cosmetic formulation according to the embodiment of the inventive concept may contain the above-described antioxidant complex. Because the antioxidant complex contained in this cosmetic formulation is substantially the same as the above-described antioxidant complex, a redundant description thereof will be omitted.

The cosmetic formulation according to an embodiment of the inventive concept may have following effects: for example, anti-aging, anti-wrinkle, elasticity improvement, moisturizing, skin barrier improvement, atopy, anti-inflammatory, acne, antibacterial, complexion improvement, skin tone improvement, pore improvement, sebum control, trouble improvement, slimming, UV protection and whitening effects.

In addition, the cosmetic formulation according to an embodiment of the inventive concept may be prepared into various formulations. Specifically, for example, the cosmetic formulation may include softening cosmetic water, nutritional cosmetic water, nutritional lotion, massage cream, nutritional cream, pack, gel or skin adhesive type cosmetic. In addition, the formulation may be a transdermal administered formation such as a lotion, ointment, gel, cream, patch or spray.

Hereinafter, the contents of the inventive concept will be described in more detail through Examples and Test Examples. These Examples are only presented to understand the content of the inventive concept, and the scope of the inventive concept is not limited to these Examples. The skilled person to the art may perform modifications, substitutions, insertions, etc. commonly known in the art, which are included within the scope of the inventive concept.

### Preparation Example 1: Complex of Glycerol and Dihydroquercetin

500 g of glycerol and 125 g of dihydroquercetin (powder, purity 98% or greater) is added to an anchor stirrer (100-150 REV/min) and slowly heated to a temperature of 70 to 90° C. while continuously stirring the mixture. The continue stirring is performed until the dihydroquercetin is completely dissolved. The solution is cooled and filtered in vacuum. As a result, a complex of glycerol and dihydroquercetin is obtained. The complex is a clear yellow sticky solution. Further, stability of the complex is maintained for 4 to 5 months, and then dihydroquercetin may precipitate.

### Preparation Example 2: Dihydroquercetin Complex Using Glycerol and L-Arginine

445 g of glycerol and 50 g of dihydroquercetin are added to an anchor stirrer (100-150 REV/min) and slowly heated to a temperature of 70 to 90° C. while stirring continuously the mixture, and the stirring is continued until the dihydroquercetin is completely dissolved. The solution is cooled to 40 to 50° C. and filtered in vacuum using a filter. Finally, 5 g of L-arginine (a substance that increases the solubility of flavonoids, solubility 15 mg/water 100 g at 21° C.) dissolved in deionized water is added to a transparent yellow sticky solution and is stirred. After cooling the solution, the solution is filtered in vacuum. As a result, a complex of glycerol and dihydroquercetin is obtained. The complex is a clear yellow sticky solution. Further, the stability of the complex is maintained for 4 to 5 months, and then, dihydroquercetin may precipitate.

### Preparation Example 3: Complex of Propylene Glycol and Dihydroquercetin

450 g of propylene glycol and 50 g of dihydroquercetin are added to an anchor stirrer (100-150 REV/min) and slowly heated to a temperature of 70 to 90° C. while stirring continuously the mixture. The continue stirring is performed until the dihydroquercetin is completely dissolved. Then, the solution is cooled and filtered in vacuum. As a result, a complex of propylene glycol and dihydroquercetin is obtained. The complex is a clear yellow sticky solution. Further, the stability of the complex is maintained. That is, even after several months, the precipitation of dihydroquercetin did not occur.

### Preparation Example 4: Complex of Butylene Glycol and Dihydroquercetin

500 g of butylene glycol and 125 g of dihydroquercetin are added to an anchor stirrer (100-150 REV/min) and slowly heated to a temperature of 80 to 90° C. while stirring continuously the mixture. The continue stirring is performed until the dihydroquercetin is completely dissolved. Then, the solution is cooled and filtered in vacuum. As a result, a complex of propylene glycol and dihydroquercetin is obtained. The complex is a clear yellow sticky solution. Further, the stability of the complex was maintained. That is, even after several months, the precipitation of dihydroquercetin did not occur.

### Experimental Example: Measurement of Antioxidant Capacity

The majority of studies evaluating the antioxidant capacity of dihydroquercetin and other antioxidants have utilized chemical-based assays.

The present inventors have studied the antioxidant capacity to reduce oxidative stress in cells using CAP-e bioassay (hereinafter, referred to as CAP-e assay).

The CAP-e assay is used to test whether natural products contain antioxidants that may protect living cells from oxidative damage. Therefore, when a certain protective effect is exhibited in the CAP-e assay, a biologically significant antioxidant protective effect is exhibited from the product. In addition, the CAP-e analysis is useful for comparing multiple production lots of the same product with each other and for comparison between capacities of different test products or raw substances.

CAP-e Bioassay

Cellular antioxidant protection (CAP-e) assay of red blood cells was used to evaluate the capacity of antioxidant complexes to protect cells from oxidative stress after the antioxidant complexes enter the cells.

Human red blood cells were treated for 20 minutes using the antioxidant complex serially diluted in physiological saline. During this incubation period, the antioxidant complexes that may pass through a cell membrane may enter the cells. After exposure of the red blood cells to the antioxidant complex which was tested twice for each dose of the antioxidant complex, the red blood cells were washed twice with PBS to remove the antioxidant complex that was not absorbed by the cells.

The red blood cells were loaded with an indicator dye DCF-DA that becomes fluorescent when oxidized, and then a peroxyl free radical generator AAPH was added thereto to induce oxidative damage to the cells. A relative fluorescence intensity of each cell culture was measured at 488 nanometers using a Tecan Spectrafluor plate reader (Tecan, Männedorf, Switzerland). A reference for oxidation at the cellular level was defined based on a low fluorescence intensity of untreated control cells (cells treated with PBS but not treated with a test product or AAPH). A positive control for cellular oxidative damage was defined as red blood cells exposed to AAPH alone under the absence of the antioxidant complex. A reduced fluorescence intensity was observed in the red blood cells exposed to the test product prior to exposure to AAPH. This was an indication that the antioxidant complex contains an antioxidant that may penetrate antigens into the red blood cells and protect the cells against AHCC induced oxidative damage.

IC50 (for CAP-e assay) is a measure of effectiveness of a compound that inhibits oxidative damage. The IC50 may be calculated when the product is strong enough to exhibit 50% or greater inhibition within the tested dose range.

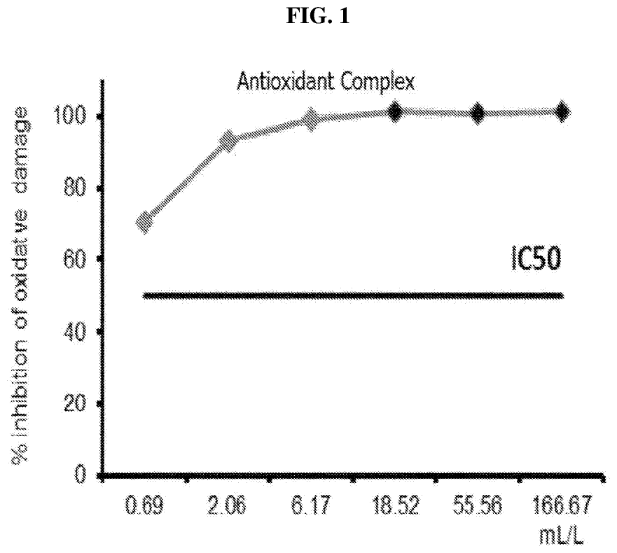

The test product was administered to achieve very high protection level at all of dosages in a standard dosage range as used. Even at the lowest dose, the protective effect was maintained at 50% or greater (FIG. 1). The result as obtained was used in a second test on this test product to fine tune a dose for optimal performance. In graphs obtained at low concentrations (Graph 2), a point on each graph where the IC50 line intersects a curve reflects the IC50 dose of the test product, i.e., the dose exhibiting 50% inhibition of the oxidative damage. This IC50 dose is compared to the IC50 dose of gallic acid (used as a control in the assay) as a known antioxidant. As a result, a CAP-e value reported in an unit corresponding to gallic acid is calculated.

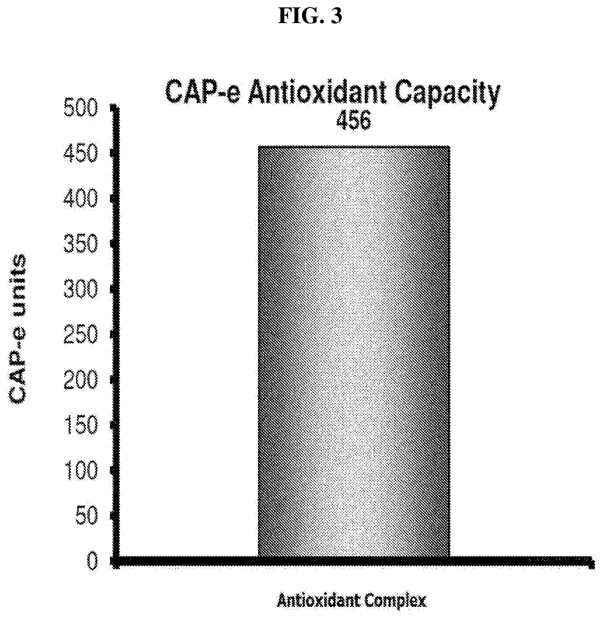

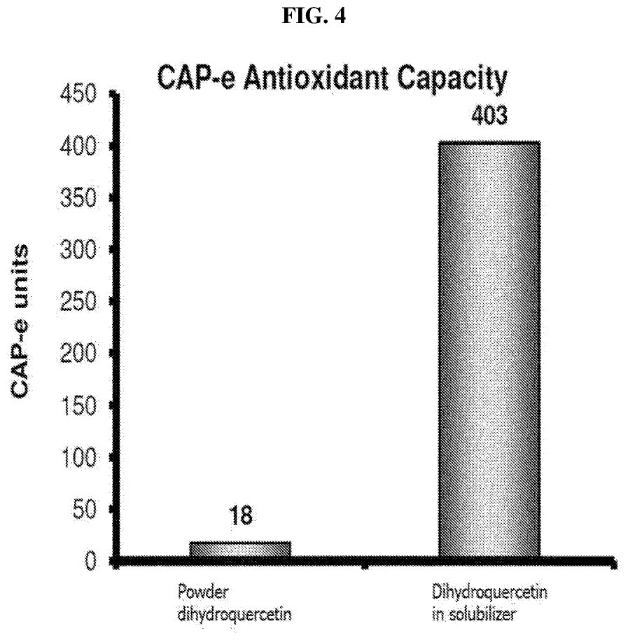

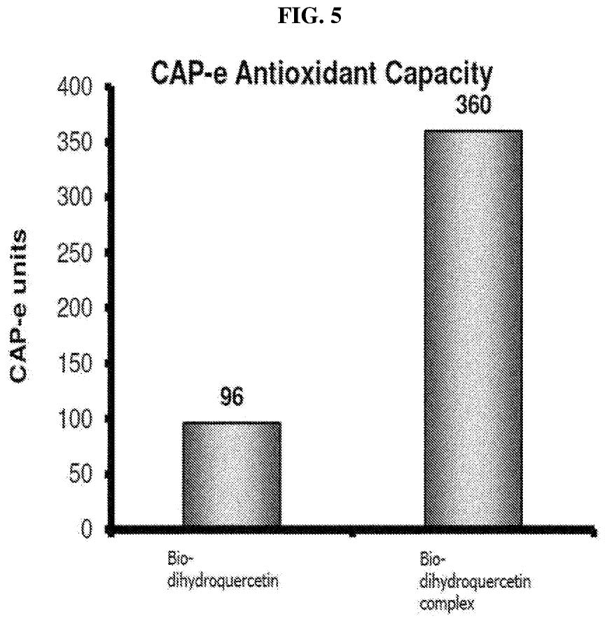

In addition to the antioxidant complex obtained according to the inventive concept (FIG. 3), CAP-e of dihydroquercetin in each of other forms was analyzed (FIGS. 4 and 5) to compare the cell protective capacity. Among them, the mixture of dihydroquercetin with the solubilizer and the antioxidant complex obtained according to the inventive concept method exhibited very strong antioxidant capacity. A dihydroquercetin powder and a biodihydroquercetin powder sold as a health supplement did not exhibit the intracellular antioxidant capacity.

To the contrary, the antioxidant complex based on biodihydroquercetin which had a relatively higher CAP-e result than that of a general dihydroquercetin powder exhibited a lower protective capacity than that of the complex based on the general dihydroquercetin.

When the general dihydroquercetin powder is introduced into physiological saline, the power is basically almost completely insoluble therein and no interaction between the compound and the cells occurs.

Therefore, the protection of antioxidants in the cells does not occur. In addition, a simple mixture between dihydroquercetin and the solubilizer is not suitable for use as a cosmetic mixture because the dihydroquercetin is not soluble in the solubilizer used in the inventive concept and is heterogeneous. The simple mixture is not preferable for a preparation method of the antioxidant complex according to the inventive concept.

To the contrary, when dihydroquercetin is present under presence of a carrier compound or a complex, the interaction thereof with the cells occurs. That is, the antioxidant complex exhibits this difference. In particular, the antioxidant complex obtained according to the inventive concept was the most powerful in the CAP-e analysis.

According to the inventive concept, an increase in the unique antioxidant properties of the dihydroquercetin is achieved. The antioxidant complex prepared by the inventive concept is distinguished from other antioxidants in other forms of dihydroquercetin in that the antioxidant complex prepared by the inventive concept a) is physiologically harmless, b) uses a cosmetic composition containing a high concentration of dihydroquercetin, c) has high biological activity, d) has strong antioxidant capacity in vivo, e) is obtained in a simple preparation process, and f) has stability in the preparation process of cosmetic products.

Most cosmetic and dermatological treatment agents are based on a lipid phase, and individual components thereof tend to be oxidized at room temperature. Thus, physicochemical properties and functional properties thereof may be completely changed. An antioxidant as a component of a cosmetic formulation has advantages of protecting the lipid component and extending a shelf life of the formulation.

The effects of the inventive concept are not limited to the above-mentioned effects, and other effects not mentioned will be clearly understood by those skilled in the art from the above description.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A composition constituting an antioxidant complex, the composition containing:
flavonoid having antioxidant efficacy; and
a solubilizer to solubilize the flavonoid.

2. The composition of claim 1, wherein the flavonoid includes dihydroquercetin,
wherein the flavonoid is a mixture of the dihydroquercetin and one selected from a group consisting of armadendrin, eriodictiol, quercetin, naringenin, and pinocembrin depending on purification of an extract of the dihydroquercetin.

3. The composition of claim 1, wherein the solubilizer is one selected from a group consisting of butylene glycol, propylene glycol and glycerol.

4. The composition of claim 1, wherein a content of the flavonoid is 0.001 to 50% by weight, and a content of the solubilizer is 50 to 99.999% by weight.

5. The composition of claim 4, wherein the content of the flavonoid is 0.1 to 20% by weight, and the content of the solubilizer is 70 to 99.9% by weight

6. The composition of claim 1, wherein the composition constituting the antioxidant complex further contains at least one selected from a group consisting of surfactant, pigment, stabilizer, emollient and humectant.

7. An antioxidant complex containing:
flavonoid having antioxidant efficacy; and
a solubilizer to solubilize the flavonoid.

8. The antioxidant complex of claim 7, wherein the flavonoid and the solubilizer are agglomerated with each other.

9. The antioxidant complex of claim 8, wherein the flavonoid and the solubilizer are agglomerated with each other such that the solubilizer surrounds an outer periphery of the flavonoid.

10. The antioxidant complex of claim 7, wherein the flavonoid includes dihydroquercetin,
wherein the flavonoid is a mixture of the dihydroquercetin and one selected from a group consisting of armadendrin, eriodictiol, quercetin, naringenin, and pinocembrin depending on purification of an extract of the dihydroquercetin.

11. The antioxidant complex of claim 7, wherein the solubilizer is one selected from a group consisting of butylene glycol, propylene glycol and glycerol.

12. The antioxidant complex of claim 7, wherein a content of the flavonoid is 0.001 to 50% by weight, and a content of the solubilizer is 50 to 99.999% by weight.

13. The antioxidant complex of claim 12, wherein the content of the flavonoid is 0.1 to 20% by weight, and the content of the solubilizer is 70 to 99.9% by weight

14. The antioxidant complex of claim 7, wherein the composition constituting the antioxidant complex further contains at least one selected from a group consisting of surfactant, pigment, stabilizer, emollient and humectant.

15. A cosmetic formulation containing an antioxidant complex, the antioxidant complex containing:
flavonoid having antioxidant efficacy; and
a solubilizer to solubilize the flavonoid.
